(19) United States
(12) Patent Application Publication
(10) Pub. No.: US 2011/0003866 A1
(43) Pub. Date: Jan. 6, 2011

(54) NOVEL 2-DIMETHYLAMINO-3-AMIDO-6-AMINO-PYRIDINE DERIVATIVES USEFUL AS POTASSIUM CHANNEL ACTIVATORS

(76) Inventors: William Dalby Brown, Soborg (DK); Carsten Jessen, Birkerod (DK); Dorte Strøbæk, Farum (DK)

Correspondence Address: BIRCH STEWART KOLASCH & BIRCH, PO BOX 747, FALLS CHURCH, VA, 22040-0747, US

(21) Appl. No.: 12/747,414

(22) PCT Filed: Dec. 10, 2008
(86) PCT No.: PCT/EP08/67161

§ 371 Date: Sep. 14, 2010

(30) Foreign Application Priority Data
Dec. 11, 2007 (DK) PA 2007 01765

(51) Int. Cl. A61K 31/44; C07D 213/75; A61P 25/00; A61P 25/28; A61P 25/06; A61P 25/18; A61P 25/08; A61P 25/22; A61P 25/24

(52) U.S. Cl. 514/352; 546/308

(57) ABSTRACT

This invention relates to novel 2-dimethylamino-3-amido-6-amino-pyridine derivatives having medical utility, to use of the 2-dimethylamino-3-amido-6-amino-pyridine derivatives of the invention for the manufacture of a medicament, to pharmaceutical compositions comprising the 2-dimethylamino-3-amido-6-amino-pyridine derivatives of the invention, and to methods of treating a disorder, disease or a condition of a subject, which disorder, disease or condition is responsive to activation of Kᵥ7 channels.

### TECHNICAL FIELD

This invention relates to novel 2-dimethylamino-3-amido-6-amino-pyridine derivatives having medical utility, to use of the 2-dimethylamino-3-amido-6-amino-pyridine derivatives of the invention for the manufacture of a medicament, to pharmaceutical compositions comprising the 2-dimethylamino-3-amido-6-amino-pyridine derivatives of the invention, and to methods of treating a disorder, disease or a condition of a subject, which disorder, disease or condition is responsive to activation of Kᵥ7 channels.

### BACKGROUND ART

Potassium (K⁺) channels are structurally and functionally diverse families of K⁺-selective channel proteins, which are ubiquitous in cells, indicating their central importance in regulating a number of key cell functions. While widely distributed as a class, K⁺ channels are differentially distributed as individual members of this class or as families.

Recently a new family of voltage gated potassium channels, the KCNQ channels, has attracted attention as target for therapeutic development. The human KCNQ1 channel was disclosed by Wang, Q et al. [Wang, Q et al.; Nature Genet. 1996 12 17-23], the human KCNQ2 channel was disclosed by Biervert et al. [Biervert et al.; Science 1998 279 403-406]; the human KCNQ3 channel was disclosed by Schroeder et al. [Schroeder et al.; Nature 1998 396 687-690]; the human KCNQ4 channel was disclosed by Kubisch et al. [Kubisch et al.; Cell 1999 96 (3) 437-46]; and the human KCNQ5 channel was disclosed by Schroeder et al. [Schroeder et al.; J. Biol. Chem. 2000 275 (31) 24089-24095]. According to the latest nomenclature KCNQ1-KCNQ5 channels now are also designated Kᵥ7.1-Kᵥ7.5.

Due to the distribution of KCNQ channels within the organism, KCNQ channel modulators are considered potentially useful for the treatment or alleviation of conditions as diverse as pain, migraine, tension type headache, CNS disorders, CNS damage caused by trauma, stroke or neurodegenerative illness or diseases, learning and cognitive disorders, motion and motor disorders, multiple sclerosis, heart failure, cardiomyopathia, cardiac disorders, inflammatory diseases, ophthalmic conditions, progressive hearing loss or tinnitus, obstructive or inflammatory airway diseases, for inducing or maintaining bladder control including the treatment or prevention of urinary incontinence.

### SUMMARY OF THE INVENTION

It is an object of the present invention to provide novel 2-dimethylamino-3-amido-6-amino-pyridine derivatives having medical utility for combating disorders, diseases or conditions responsive to activation of Kᵥ7 channels.

In its first aspect the invention provides 2-dimethylamino-3-amido-6-amino-pyridine derivatives of Formula I

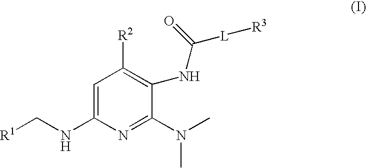

a stereoisomer or a mixture of its stereoisomers, or a pharmaceutically-acceptable addition salt thereof, or an N-oxide thereof, wherein R¹, R², R³ and L are as defined below.

In another aspect the invention provides pharmaceutical compositions comprising a therapeutically effective amount of the 2-dimethylamino-3-amido-6-amino-pyridine derivative of the invention, or a pharmaceutically-acceptable addition salt thereof.

Viewed from a third aspect the invention relates to the use of the 2-dimethyl-amino-3-amido-6-amino-pyridine derivative of the invention, or a pharmaceutically-acceptable addition salt thereof, for the manufacture of pharmaceutical compositions.

In a fourth aspects the invention provides a method of treatment, prevention or alleviation of a disease or a disorder or a condition of a living animal body, including a human, which disorder, disease or condition is responsive to activation of Kᵥ7 channels, which method comprises the step of administering to such a living animal body in need thereof, a therapeutically effective amount of the 2-dimethylamino-3-amido-6-amino-pyridine derivative of the invention, or a pharmaceutically-acceptable addition salt thereof.

Other objects of the invention will be apparent to the person skilled in the art from the following detailed description and examples.

### DETAILED DISCLOSURE OF THE INVENTION

The 2-dimethylamino-3-amido-6-amino-pyridine derivatives of the invention may be characterised by Formula I

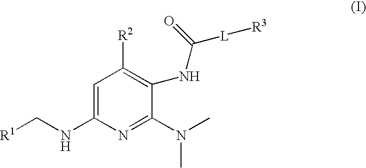

a stereoisomer or a mixture of its stereoisomers, or a pharmaceutically-acceptable addition salt thereof, or an N-oxide thereof, wherein
R¹ represents alkyl or phenyl, which phenyl is optionally substituted one or more times with substituents selected from alkyl, halo and trifluoromethyl;
R² represents hydrogen;
R³ represents alkyl or phenyl, which phenyl is optionally substituted one or more times with substituents selected from alkyl, halo, alkoxy and trifluoromethyl; and
L represents a linker selected from —CR′R″—, —CH₂—CR′R″—, —CR′R″—CH₂— and cycloalkyl, wherein R′ and R″, independently of each other, represent hydrogen, alkyl or halo.

In one embodiment the derivative of the invention is a compound of Formula I, or a pharmaceutically-acceptable addition salt thereof, or an N-oxide thereof, wherein
R¹ represents alkyl or phenyl, which phenyl is optionally substituted one or more times with substituents selected from alkyl, halo and trifluoromethyl; R² represents hydrogen; R³ represents phenyl, which phenyl is optionally substituted one or more times with substituents selected from alkyl, halo and trifluoromethyl; and L represents a linker selected from —CR′R″—, —CH₂—CR′R″—, —CR′R″—CH₂— and cycloalkyl, wherein R′ and R″, independently of each other, represent hydrogen, alkyl or halo.

In another embodiment the derivative of the invention is a compound of Formula I, or a pharmaceutically-acceptable addition salt thereof, or an N-oxide thereof, wherein R¹ represents alkyl or phenyl, which phenyl is optionally substituted one or more times with substituents selected from alkyl, halo and trifluoromethyl.

In another embodiment R¹ represents phenyl, which phenyl is optionally substituted one or more times with substituents selected from alkyl, halo and trifluoromethyl.

In another embodiment R¹ represents a phenyl group substituted one or more times with substituents selected from alkyl, halo and trifluoromethyl.

In another embodiment R¹ represents a phenyl group substituted one or more times with alkyl.

In another embodiment R¹ represents phenyl substituted once or twice with halo e.g. fluoro.

In another embodiment R¹ represents phenyl substituted once with halo, e.g. fluoro.

In another embodiment R¹ represents alkyl.

In another embodiment the derivative of the invention is a compound of Formula I, or a pharmaceutically-acceptable addition salt thereof, or an N-oxide thereof, wherein R² represents hydrogen.

In another embodiment the derivative of the invention is a compound of Formula I, or a pharmaceutically-acceptable addition salt thereof, or an N-oxide thereof, wherein R³ represents alkyl.

In another embodiment the derivative of the invention is a compound of Formula I, or a pharmaceutically-acceptable addition salt thereof, or an N-oxide thereof, wherein R³ represents phenyl optionally substituted one or more times with substituents selected from alkyl, halo, alkoxy and trifluoromethyl.

In another embodiment R³ represents phenyl, optionally substituted one or more times with substituents selected from alkyl, halo and trifluoromethyl In another embodiment R³ represents phenyl substituted one or more times with substituents selected from alkyl, halo, alkoxy and trifluoromethyl.

In another embodiment R³ represents phenyl substituted one or two times with substituents selected from halo, e.g. fluoro, and trifluoromethyl.

In another embodiment R³ represents phenyl substituted once or twice with alkyl.

In another embodiment R³ represents phenyl substituted once or twice with alkoxy.

In another embodiment R³ represents phenyl substituted once or twice with halo, e.g. fluoro.

In another embodiment R³ represents phenyl substituted once with halo, e.g. fluoro.

In another embodiment R³ represents phenyl substituted twice with halo, e.g. fluoro.

In another embodiment R³ represents phenyl substituted once or twice with trifluoromethyl.

In another embodiment R³ represents phenyl substituted once with halo, e.g. fluoro, and once with trifluoromethyl.

In another embodiment R³ represents phenyl.

In another embodiment the derivative of the invention is a compound of Formula I, or a pharmaceutically-acceptable addition salt thereof, or an N-oxide thereof, wherein L represents a linker selected from —CR′R″—, —CH₂—CR′R″—, —CR′R″—CH₂— and cycloalkyl, wherein R′ and R″, independently of each other, represent hydrogen, alkyl or halo.

In another embodiment L represents a linker selected from —CR′R″—, —CH₂—CR′R″— and cycloalkyl, wherein R′ and R″, independently of each other, represent hydrogen or alkyl, e.g. methyl.

In another embodiment L represents a linker selected from —CH₂—, —CH₂—CH₂—, —CH₂—CH₂—CH₂—, —CH(CH₃)—, —CH₂—CH(CH₃)—, —CH₂—C(CH₃)₂— and cyclopropyl.

In another embodiment L represents —CH₂—.

In another embodiment L represents —CH₂—CH₂—.

In another embodiment L represents —CH₂—CH₂—CH₂—.

In another embodiment L represents —CH(CH₃)—.

In another embodiment L represents —CH₂—CH(CH₃)—.

In another embodiment L represents —CH₂—C(CH₃)₂—.

In another embodiment L represents cyclopropyl.

In another embodiment the derivative of the invention is a compound of Formula I, or a pharmaceutically-acceptable addition salt thereof, or an N-oxide thereof, wherein R¹ represents phenyl substituted once with halo, e.g. fluoro, R³ represents phenyl optionally substituted one or more times with halo or trifluoromethyl, and L represents —CH₂—, —CH(CH₃—)—, —CH₂—CH(CH₃)— or cyclopropyl.

In another embodiment the derivative of the invention is a compound of Formula I, or a pharmaceutically-acceptable addition salt thereof, or an N-oxide thereof, wherein R¹ represents phenyl substituted once with halo, e.g. fluoro, R³ represents alkyl, and L represents —CH₂—.

In another embodiment the derivative of the invention is a compound of Formula I, or a pharmaceutically-acceptable addition salt thereof, or an N-oxide thereof, wherein R¹ represents phenyl substituted one or more times with alkyl, R³ represents phenyl substituted once or twice with halo, e.g. fluoro, and L represents —CH₂—.

In another embodiment the 2-dimethylamino-3-amido-6-amino-pyridine derivative of the invention is:
N-[2-Dimethylamino-6-(4-fluoro-benzylamino)-pyridin-3-yl]-2-(3,4-difluoro-phenyl)-acetamide;
N-[2-Dimethylamino-6-(4-fluoro-benzylamino)-pyridin-3-yl]-2-(3,5-difluoro-phenyl)-acetamide;
N-[2-Dimethylamino-6-(4-fluoro-benzylamino)-pyridin-3-yl]-2-(3-fluoro-4-trifluoromethyl-phenyl)-acetamide;
(S)—N-[2-Dimethylamino-6-(4-fluoro-benzylamino)-pyridin-3-yl]-2-phenyl-propionamide;
N-[2-Dimethylamino-6-(4-fluoro-benzylamino)-pyridin-3-yl]-3-(3-fluoro-phenyl)-propionamide;
trans-2-Phenyl-cyclopropanecarboxylic acid[2-dimethylamino-6-(4-fluoro-benzyl-amino)-pyridin-3-yl]-amide;
N-[2-Dimethylamino-6-(4-fluoro-benzylamino)-pyridin-3-yl]-2-(3-fluoro-phenyl)-acetamide;
N-[2-Dimethylamino-6-(4-fluoro-benzylamino)-pyridin-3-yl]-3,3-dimethyl-butyramide;
   or a pharmaceutically-acceptable addition salt thereof.

In another embodiment the 2-dimethylamino-3-amido-6-amino-pyridine derivative of the invention is:
2-(3,5-Difluoro-phenyl)-N-[2-dimethylamino-6-(2,4,6-trimethyl-benzylamino)-pyridin-3-yl]acetamide;
2-(3,5-Difluoro-phenyl)-N-[2-dimethylamino-6-(2,6-dimethyl-benzylamino)-pyridin-3-yl]-acetamide;
N-[2-Dimethylamino-6-(5-fluoro-2-methyl-benzylamino)-pyridin-3-yl]-3,3-dimethyl-butyramide;
   or a pharmaceutically-acceptable addition salt thereof.

Any combination of two or more of the embodiments described herein is considered within the scope of the present invention.

### Definition of Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates the radical —O-alkyl. Representative examples are methoxy, ethoxy, propoxy (e.g. 1-propoxy, 2-propoxy), butoxy (e.g. 1-butoxy, 2-butoxy, 2-methyl-2-propoxy), pentoxy (1-pentoxy, 2-pentoxy), hexoxy (1-hexoxy, 3-hexoxy), and the like.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

In the context of this invention halo represents fluoro, chloro, bromo or iodo.

### Pharmaceutically Acceptable Salts

The 2-dimethylamino-3-amido-6-amino-pyridine derivatives of the invention may be provided in any form suitable for the intended administration. Suitable forms include pharmaceutically (i.e. physiologically) acceptable salts, and pre- or prodrug forms of the 2-dimethylamino-3-amido-6-amino-pyridine derivatives of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzene-sulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

### Steric Isomers

The 2-dimethylamino-3-amido-6-amino-pyridine derivatives of the present invention may exist in (+) and (−) forms as well as in racemic forms (±). The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Yet another method for resolving racemates is by covalent introduction of an additional steric center. Separation upon chromatography on a non-chiral matrix or simple crystallisation followed by cleavage of the covalent bond used for introducing yet another chiral center will liberate the resolved material. Racemic compounds of the present invention can thus be resolved into their optical antipodes, e.g., by fractional crystallisation of d- or l- (tartrates, mandelates, or camphorsulphonate) salts for example or by covalent modifications.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Methods of Preparation

The 2-dimethylamino-3-amido-6-amino-pyridine derivatives of the present invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The 2-dimethylamino-3-amido-6-amino-pyridine derivatives of the present invention have been found useful as modulators of the voltage gated Kᵥ7 (KCNQ) potassium ion channels. At present five such channels are known, i.e. the Kᵥ7.1 (KCNQ1) channel, the Kᵥ7.2 (KCNQ2) channel, the Kᵥ7.3 (KCNQ3) channel, the Kᵥ7.4 (KCNQ4) channel, and the Kᵥ7.5 (KCNQ5) channel, and heteromeric combinations of these subunits. Moreover, the modulatory activity may be inhibitory (i.e. inhibitory activity) or stimulatory (i.e. activating activity).

The modulatory activity may be determined using conventional methods, e.g. binding or activity studies, known in the art, e.g. as described in WO 2004/080377 (NeuroSearch A/S) or as described in the working examples.

In one aspect of the invention, the compounds of the invention show stimulating activity at Kᵥ7.2, Kᵥ7.3, Kᵥ7.4 and/or Kᵥ7.5 potassium channels, and heteromeric combinations hereof.

Accordingly, the compounds of the invention are considered useful for the treatment, prevention or alleviation of a disease or a disorder or a condition of a living animal body, including a human, which disorder, disease or condition is responsive to modulation of a Kᵥ7 potassium channel.

Due to the distribution of Kᵥ7 channels within the organism, Kᵥ7 channel modulators are considered useful for the treatment or alleviation of conditions as diverse as an affective disorder, a neuro-physiological disorder, an anxiety disorder, depression, a bipolar disorder, a sleep disorder, addiction, an eating disorder, a phobia, a neurodegenerative disorder, Parkinson's disease, a mood disorder, a psychotic disorder, a compulsive behaviour, mania, psychosis, schizophrenia, dementia, Alzheimer's disease, epilepsy, convulsions, seizure disorders, absence seizures, vascular spasms, coronary artery spasms, tremor, muscle spasms, myasthenia gravis, a motor neuron disease, motion and motor disorders, a tic disorder, a Parkinson-like motor disorder, essential tremors, multiple sclerosis, amyelotrophic lateral sclerosis (ALS), multiple system atrophy, corticobasal degeneration, HIV associated dementia, Huntington's disease, Pick's disease, torsades de pointes, functional bowel disorders, CNS damage caused by trauma, stroke or neurodegenerative illness or diseases, ataxia, myokymia, spasticity, myopathy, learning and cognitive disorders, memory dysfunction, memory impairment, age-associated memory loss, Down's syndrome, pain, acute or chronic pain, mild pain, moderate or severe pain, neuropathic pain, central pain, pain related to diabetic neuropathy, to postherpetic neuralgia, to peripheral nerve injury, somatic pain, visceral pain or cutaneous pain, pain caused by inflammation or by infection, postoperative pain, phantom limb pain, neuronal hyperexcitability disorders, peripheral nerve hyperexcitability, chronic headache, migraine, migraine-related disorders, tension-type headache, hypotension, hypertension, heart failure, cardiac disorders, cardiomyopathia, cardiac arrhythmia, cardiac ischaemia, long QT syndrome, inflammatory diseases or conditions, inflammatory bowel disease, Crohn's disease, ulcerative colitis, Creutzfeld-Jacobs disease, an obstructive or inflammatory airway disease, asthma, an airway hyper reactivity, pneumoconiosis, aluminosis, anthracosis, asbestosis, chalicosis, ptilosis, siderosis, silicosis, tabacosis, byssinosis, chronic obstructive pulmonary disease (COPD), excerbation of airways hyper reactivity, cystic fibrosis, hearing impairment or hearing loss, progressive hearing loss, tinnitus, a drug-dependence or drug-addiction disorder, hyperactive gastric motility, ophthalmic conditions, erectile dysfunction, fibromylgia, for inducing or maintaining bladder control, nocturia, bladder spasms, overactive bladder (OAB), bladder outflow obstruction, interstitial cystitis (IC) (also called painfull bladder syndrome) and urinary incontinence.

In another embodiment the disease, disorder or condition contemplated according to the invention is an anxiety disorder such as panic disorder, agora-phobia, phobias, social anxiety disorder, obsessive-compulsive disorder and post-traumatic stress disorder. In another embodiment the disease, disorder or condition contemplated according to the invention is anxiety. In another embodiment the disease, disorder or condition contemplated according to the invention is schizophrenia.

In one embodiment the compounds of the invention are considered useful for treatment, prevention or alleviation of a disease, disorder or adverse condition of the CNS. In another embodiment, the disease, disorder or condition is an affective disorder, a neuro-physiological disorder, an anxiety disorder, depression, a bipolar disorder, a sleep disorder, addiction, an eating disorder, a phobia, a neurodegenerative disorder, Parkinson's disease, a mood disorder, a psychotic disorder, a compulsive behaviour, mania, psychosis or schizophrenia.

In another embodiment the compounds of the invention are considered useful for treatment, prevention or alleviation of a CNS damage caused by trauma or by a spinal cord damage, stroke, traumatic brain injury, a neurodegenerative illness or disease, dementia, Alzheimer's disease, a motor neuron disease, a Parkinson-like motor disorder, essential tremors, multiple sclerosis, amyelotrophic lateral sclerosis (ALS), multiple system atrophy, HIV associated dementia, Huntington's disease, Pick's disease, torsades de pointes, tremor, muscle spasms, myasthenia gravis, convulsions, ataxia, myokymia, seizures, epilepsy or spasticity. In another embodiment the compounds of the invention are useful for the treatment, prevention or alleviation of epilepsy.

In another embodiment the compounds of the invention are considered useful for treatment, prevention or alleviation of pain, including acute and chronic pain, mild pain, moderate or even severe pain of acute, chronic or recurrent character, as well as postoperative pain, phantom limb pain, chronic headache, post therapeutic neuralgia, neuropathic pain, central pain, or pain related to diabetic neuropathy, to postherpetic neuralgia, to peripheral nerve injury or drug addiction, migraine and migraine-related disorders and to tension-type headache.

In another embodiment the pain is somatic pain, incl. visceral pain or cutaneous pain, or pain caused by inflammation or by infection. In another embodiment the pain is neuropathic, e.g. caused by injury to the central or peripheral nervous system, e.g. due to tissue trauma, infection, diabetes, an autoimmune disease, arthritis or neuralgia. In another embodiment the compounds of the invention are useful for the treatment, prevention or alleviation of pain and neuropathic pain.

In another embodiment the compounds of the invention are considered useful for treatment, prevention or alleviation of addiction, e.g. drug addiction, drug abuse, cocaine abuse, nicotine abuse, tobacco abuse, alcohol addiction or alcoholism, or withdrawal symptoms caused by the termination of abuse of chemical substances, in particular opioids, heroin, cocaine and morphine, benzodiazepines and benzodiazepine-like drugs, and alcohol.

In another embodiment the compounds of the invention are considered useful for treatment, prevention or alleviation of a learning and cognitive disorder, memory dysfunction, memory impairment, age-associated memory loss or Down's syndrome.

In another embodiment the compounds of the invention are considered useful for treatment, prevention or alleviation of chronic headache, migraine, migraine-related disorders or tension-type headache. In another embodiment the compounds of the invention are considered useful for treatment or alleviation of migraine.

In another embodiment the compounds of the invention are considered useful for treatment, prevention or alleviation of a disease, disorder or condition associated with the heart or skeletal muscle, heart failure, cardiomyopathia, cardiac arrhythmia, cardiac ischaemia or long QT syndrome.

In another embodiment the compounds of the invention are considered useful for treatment, prevention or alleviation of an inflammatory disease or condition, inflammatory bowel disease, Crohn's disease, ulcerative colitis or Creutzfeld-Jacobs disease.

In another embodiment the compounds of the invention are considered useful for treatment, prevention or alleviation of asthma, an obstructive or inflammatory airway disease, an airway hyper reactivity, a pneumoconiosis such as aluminosis, anthracosis, asbestosis, chalicosis, ptilosis, siderosis, silicosis, tabacosis and byssinosis, a chronic obstructive pulmonary disease (COPD), excerbation of airways hyper reactivity or cystic fibrosis. In another embodiment the compounds of the invention are considered useful for treatment, prevention or alleviation of asthma.

In another embodiment the compounds of the invention are considered useful for treatment, prevention or alleviation of progressive hearing loss or tinnitus.

In another embodiment the compounds of the invention are considered useful for treatment, prevention or alleviation of an ophthalmic disorder, a drug-dependence or drug-addiction disorder or hyperactive gastric motility.

In another embodiment the compounds of the invention are considered useful for treatment, prevention or alleviation of nocturia, bladder spasms, overactive bladder (OAB), interstitial cystitis (IC) and urinary incontinence. In another embodiment the compounds of the invention are considered useful for treatment, prevention or alleviation of urinary incontinence.

### Pharmaceutical Compositions

Viewed from one aspect the invention relates to the use of a 2-dimethylamino-3-amido-6-amino-pyridine derivative of the invention, or a pharmaceutically-acceptable addition salt thereof, for the manufacture of a pharmaceutical composition for the treatment, prevention or alleviation of a disease or a disorder or a condition of a mammal, including a human, which disease, disorder or condition is responsive to modulation of Kᵥ7 channels.

Viewed from another aspect, the invention provides pharmaceutical compositions comprising a therapeutically-effective amount of a 2-dimethylamino-3-amido-6-amino-pyridine derivative of the invention, or a pharmaceutically-acceptable addition salt thereof, together with at least one pharmaceutically-acceptable carrier or diluent, for the treatment, prevention or alleviation of a disease or a disorder or a condition that is responsive to modulation of Kᵥ7 channels.

While a 2-dimethylamino-3-amido-6-amino-pyridine derivative for use according to the invention may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising a 2-dimethylamino-3-amido-6-amino-pyridine derivative of the invention, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route which suite the desired therapy. Preferred routes of administration include oral administration, in particular in tablet, in capsule, in dragé in powder, or in liquid form, and parenteral administration, in particular cutaneous, subcutaneous, intramuscular, or intravenous injection. The pharmaceutical composition may be prepared by the skilled person using standard and conventional techniques appropriate for the desired formulation. When desired, compositions adapted to give sustained release of the active ingredient may be employed.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to activation of Kᵥ7 channels, and which method comprises administering to such a living animal body, including a human, in need thereof an effective amount of a 2-dimethylamino-3-amido-6-amino-pyridine derivative of the invention.

The preferred medical indications contemplated according to the invention are those stated above.

It is at present contemplated that suitable dosage ranges are 0.1 to 2000 milligrams daily, 10-1000 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 30 mg/kg i.v. and 500 mg/kg p.o. Preferred ranges are from about 0.001 to about 100 mg/kg i.v. and from about 0.1 to about 30 mg/kg p.o.

### Examples

The invention is further illustrated with reference to the following examples, which are not intended to be in any way limiting to the scope of the invention as claimed.

The abbreviations as used in the examples have the following meaning:
MeCN: Acetonitrile
DCM: Dichloromethane
EDC.HCl: 1-ethyl-3-(3-dimethylaminopropyl)carbodiimide,HCl))
DMF: N,N-dimethylformamide
DMSO: Dimethylsulfoxide
EtOH: Ethanol
EtOAc: Ethyl acetate
HOAt: 1-Hydroxy-7-azabenzotriazole
MeOH: Methanol
NMP: N-Methylpyrrolidinone
THF: Tetrahydrofuran
RT: room temperature

### Example 1

### Preparative Example 1

The compounds of the invention may be synthesised as outlined in general terms and described in more details below.

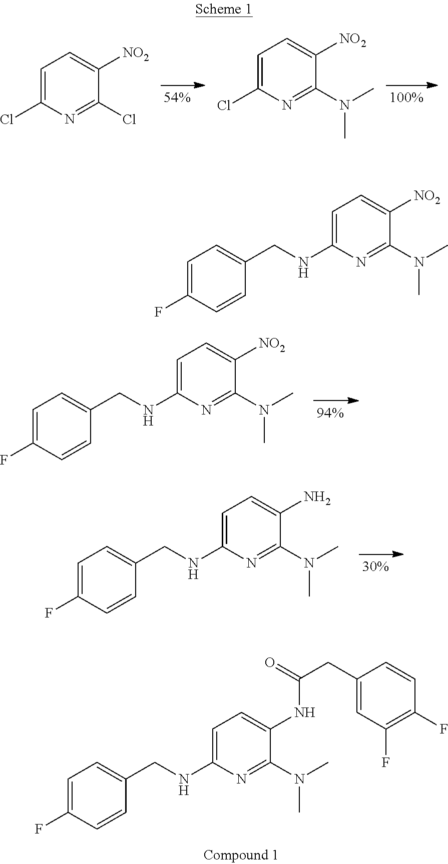

### 6-Chloro-2-(dimethylamino)-3-nitro-pyridin (intermediate compound)

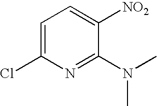

To a solution of 2,6-dichloro-3-nitropyridine (25 g, 116.6 mmol) in acetonitrile (250 mL) was added triethylamine (50 mL). The reaction mixture was cooled to 0° C. and dimethylamine hydrochloride (9.6 g, 116.6 mmol) was added portionwise. After full addition the reaction mixture was allowed to warm to room temperature.

The reaction mixture was filtrated and the isolated solid rinsed with DCM (3×100 mL). The filtrate was concentrated to ca. 250 mL and washed with 1 N HCl (2×50 mL) and brine (1×100 mL), dried on Na₂SO₄ and evaporated. Column chromategraphy (Eluent: Heptane:EtOAc 9:1) gave 12.6 g (54%) solid of the title compound with a LC-MS purity >95%. The compound was used as such in the next reaction.

### 2-Dimethylamino-6-(4-fluoro-benzylamino)-3-nitro-pyridine (intermediate compound)

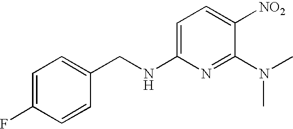

To a solution of 6-chloro-2-(dimethylamino)-3-nitro-pyridin (10.0 g, 49.6 mmol) in MeCN (100 mL) was added triethylamine (21 mL; 148.8 mmol) and 4-fluorobenzylamine (6.24 mL, 54.6 mmol. The reaction mixture was stirred over night at RT after which more 4-fluorobenzylamine (6.24 mL, 54.6 mmol) was added and the reaction mixture was stirred at reflux over night. After refluxing over night the complete reaction mixture was poured out in 150 mL 1M HCl. The waterlayer was extracted with 3×80 mL EtOAc. The combined organic layers were washed with brine, dried over Na₂SO₄ and concentrated in vacuo to yield 15.6 g (quantitative yield) with a LC-MS purity of 89% which was used as such in the next reaction.

### 2-Dimethylamino-6-(4′-fluoro-benzylamino)-3-aminopyridine (intermediate compound)

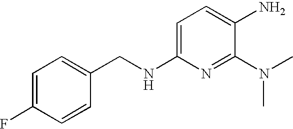

2-Dimethylamino-6-(4-fluoro-benzylamino)-3-nitro-pyridine (3.3 g; 11.4 mmol) was dissolved in 96% EtOH (50 mL) and Raney nickel catalyst 50% slurry in water (1.0 g) is added (1 mL). The reaction mixture was purged with hydrogen and a balloon of hydrogen attached. The reaction mixture was stirred vigorously at room temperature overnight after which the reaction mixture was filtered through Hyflo™ and evaporated in vacuo to yield 2.77 g as a black oil with a LC-MS purity of 74%. The product was quickly used as such in the next reaction.

In a similar manner was synthesized the following compounds:
2-Dimethylamino-6-(2′,4′,6′-trimethylbenzylamino)-3-aminopyridine (intermediate compound)
2-Dimethylamino-6-(2′,6′-dimethylbenzylamino)-3-aminopyridine (intermediate compound)
N-[2-Dimethylamino-6-(4-fluoro-benzylamino)-pyridin-3-yl]-2-(3,4-difluoro-phenyl)-acetamide (Compound 1)

2-Dimethylamino-6-(4′-fluoro-benzylamino)-3-aminopyridine (0.1; 0.38 mmol) was dissolved in DCM (4 mL) and 3,4-difluorophenylacetic acid (0.71 mg; 0.4 mmol), HOAt (74 mg; 0.54 mmol) and EDC.HCl (104 mg; 0.54 mmol). The reaction mixture was stirred overnight at room temperature. Water (3 mL) was added to the reaction mixture and the mixture was stirred vigorously for 30 min. The reaction mixture was filterd over a phase separator and the solvents were evaporated to dryness to give a solid which was purified using preparative LC-MS to give 47 mg (30%) of the title compound.

The following compounds were synthesized in a similar manner.
N-[2-Dimethylamino-6-(4-fluoro-benzylamino)-pyridin-3-yl]-2-(3,5-difluoro-phenyl)-acetamide (Compound 2)
Yield 7% over 2 steps
N-[2-Dimethylamino-6-(4-fluoro-benzylamino)-pyridin-3-yl]-2-(3-fluoro-4-trifluoromethyl-phenyl)-acetamide (Compound 3)
Yield 54% over 2 steps
(S)—N-[2-Dimethylamino-6-(4-fluoro-benzylamino)-pyridin-3-yl]-2-phenyl-propionamide (Compound 4)
Yield 33% over 2 steps
N-[2-Dimethylamino-6-(4-fluoro-benzylamino)-pyridin-3-yl]-3-(3-fluoro-phenyl)-propionamide (Compound 5)
Yield 29% over 2 steps
trans-2-Phenyl-cyclopropanecarboxylic acid[2-dimethylamino-6-(4-fluoro-benzylamino)-pyridin-3-yl]-amide (Compound 6)
Yield 24% over 2 steps
N-[2-Dimethylamino-6-(4-fluoro-benzylamino)-pyridin-3-yl]-2-(3-fluoro-phenyl)-acetamide (Compound 7)
Yield 30% over 2 steps
N-[2-Dimethylamino-6-(4-fluoro-benzylamino)-pyridin-3-yl]-3,3-dimethyl-butyramide (Compound 8)
Yield 12% over 2 steps
2-(3,5-Difluoro-phenyl)-N-[2-dimethylamino-6-(2,4,6-trimethyl-benzylamino)-pyridin-3-yl]-acetamide (Compound 9)
Yield 7% over 2 steps
2-(3,5-Difluoro-phenyl)-N-[2-dimethylamino-6-(2,6-dimethyl-benzylamino)-pyridin-3-yl]-acetamide (Compound 10)
Yield 28% over 2 steps
N-[2-Dimethylamino-6-(5-fluoro-2-methyl-benzylamino)-pyridin-3-yl]-3,3-dimethyl-butyramide (Compound 11)
Yield 72% over 2 steps.

### 2-Dimethylamino-6-(3′-fluor-6′-methyl-benzylamino)-3-aminopyridine (intermediate compound)

To a solution of 2-Dimethylamino-6-(3′-fluor-6′-methyl-benzylamino)-3-nitropyridine (prepared as in above example; 6 g; 16 mmol) in THF (100 mL) was added Raney Nickel catalyst (50% in H₂O; 1.4 g; 16 mmol) and hydrazine monohydrate (2.4 mL; 48 mmol). The reaction mixture was stirred under an argon atmosphere over night twice adding more hydrazine monohydrate (2×1 mL; 40 mmol). The reaction was filtered through a plug of Hyflo™ and Na₂SO₄ and purged with argon. The reaction was immediately used as such in the next reaction to prepare Compound 11.

| | | | LC-ESI- | |
|---|---|---|---|---|
| | | | HRMS | LC-ESI- |
| | | | Meas. | HRMS |
| No | Structure | Name | (Da) | Calc. (Da) |
| 1 | 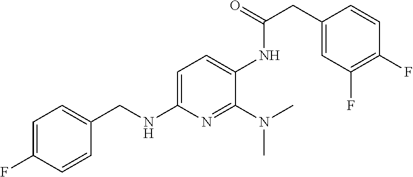 | N-[2-Dimethylamino-6-(4- fluoro-benzylamino)-pyridin-3- yl]-2-(3,4-difluoro-phenyl)- acetamide | 415.1762 | 415.1745 |
| 2 | 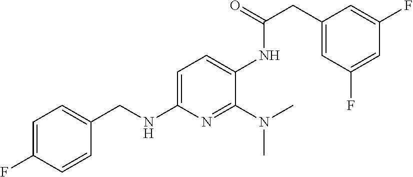 | N-[2-Dimethylamino-6-(4- fluoro-benzylamino)-pyridin-3- yl]-2-(3,5-difluoro-phenyl)- acetamide | 415.1758 | 415.1745 |
| 3 | 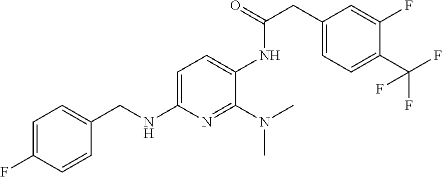 | N-[2-Dimethylamino-6-(4- fluoro-benzylamino)-pyridin-3- yl]-2-(3-fluoro-4-trifluoromethyl- phenyl)-acetamide | 465.1735 | 465.1713 |
| 4 | 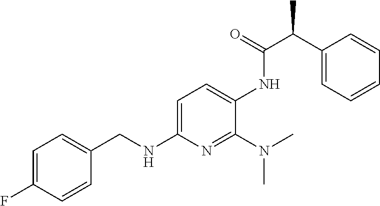 | (S)-N-[2-Dimethylamino-6-(4- fluoro-benzylamino)-pyridin-3- yl]-2-phenyl-propionamide | | |
| 5 | 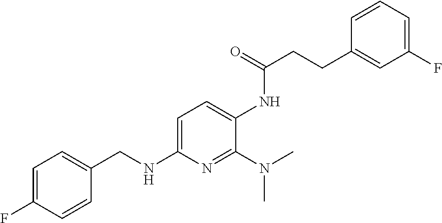 | N-[2-Dimethylamino-6-(4- fluoro-benzylamino)-pyridin-3- yl]-3-(3-fluoro-phenyl)- propionamide | 411.2004 | 411.1996 |
| 6 | 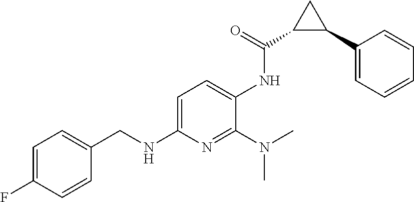 | trans-2-Phenyl-cyclopropane- carboxylic acid [2-dimethyl- amino-6-(4-fluoro-benzyl- amino)-pyridin-3-yl]-amide | 405.2104 | 405.2090 |
| 7 | 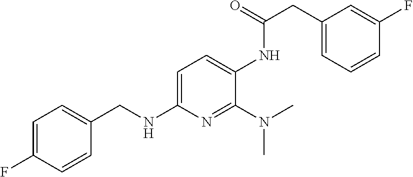 | N-[2-Dimethylamino-6-(4- fluoro-benzylamino)-pyridin-3- yl]-2-(3-fluoro-phenyl)- acetamide | 397.185 | 397.1839 |
| 8 | 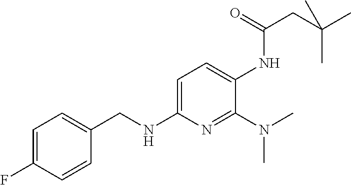 | N-[2-Dimethylamino-6-(4- fluoro-benzylamino)-pyridin-3- yl]-3,3-dimethyl-butyramide | | |
| 9 | 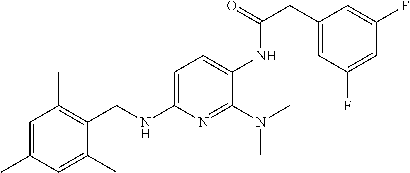 | 2-(3,5-Difluoro-phenyl)-N-[2- dimethylamino-6-(2,4,6- trimethyl-benzylamino)-pyridin- 3-yl]-acetamide | 439.229 | 439.2309 |
| 10 | 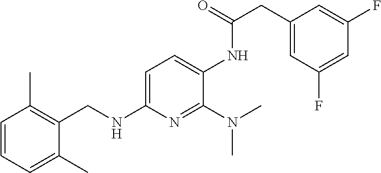 | 2-(3,5-Difluoro-phenyl)-N-[2- dimethylamino-6-(2,6-dimethyl- benzylamino)-pyridin-3-yl]- acetamide | 425.2156 | 425.2152 |
| 11 | 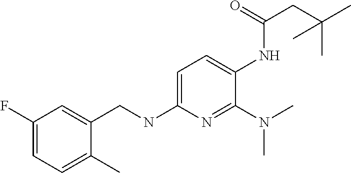 | N-[2-Dimethylamino-6-(5- fluoro-2-methyl-benzylamino)- pyridin-3-yl]-3,3-dimethyl- butyramide | 373.2416 | 373.2403 |

### Biological Activity

In a standard patch-clamp set-up, e.g. as outlined in International Patent Publication WO 2004/080377, using HEK293 cell lines stably expressing the human Kᵥ7₂₊₃ channels, the compounds of the invention were found to be activators of the channels at various concentrations at various degrees.

The effect obtained by these channel activators is described as a percentage increase in baseline current at a given concentration. The baseline current is defined as 100%, and an increase in current is expressed relative to this, i.e. an increase from 1 nA to 1.2 nA is reported as 120%.

| | I_{K} (%) |
|---|---|
| Test | conc. 0.03 μM, |
| Compound | −30 mV, 20 ms |
| 1 | 215 |
| 3 | 162 |
| 5 | 174 |
| 8 | 233 |

From the foregoing it will be appreciated that, although specific embodiments of the invention have been described herein for purposes of illustration, various modifications may be made without deviating from the spirit and scope of the invention. Accordingly, the invention is not to be limited as by the appended claims.

The features disclosed in the foregoing description, in the claims and/or in the accompanying drawings, may both separately and in any combination thereof, be material for realising the invention in diverse forms thereof.

1. A 2-dimethylamino-3-amido-6-amino-pyridine derivative of Formula I 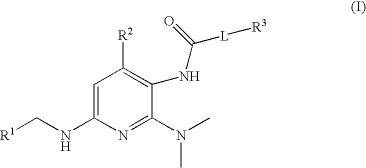
a stereoisomer or a mixture of its stereoisomers, or a pharmaceutically-acceptable addition salt thereof, or an N-oxide thereof, wherein
R¹ represents alkyl or phenyl, which phenyl is optionally substituted one or more times with substituents selected from alkyl, halo and trifluoromethyl;
R² represents hydrogen;
R³ represents alkyl or phenyl, which phenyl is optionally substituted one or more times with substituents selected from alkyl, halo, alkoxy and trifluoromethyl; and
L represents a linker selected from —CR′R″—, —CR′R″—CH₂— and cycloalkyl, wherein R′ and R″, independently of each other, represent hydrogen, alkyl or halo.

2. The 2-dimethylamino-3-amido-6-amino-pyridine derivative according to claim 1, or a stereoisomer or a mixture of its stereoisomers, or a pharmaceutically-acceptable addition salt thereof, or an N-oxide thereof, wherein R¹ represents alkyl.

3. The 2-dimethylamino-3-amido-6-amino-pyridine derivative according to claim 1, or a stereoisomer or a mixture of its stereoisomers, or a pharmaceutically-acceptable addition salt thereof, or an N-oxide thereof, wherein R¹ represents phenyl, which phenyl is optionally substituted one or more times with substituents selected from alkyl, halo and trifluoromethyl.

4. The 2-dimethylamino-3-amido-6-amino-pyridine derivative according to claim 1, or a stereoisomer or a mixture of its stereoisomers, or a pharmaceutically-acceptable addition salt thereof, or an N-oxide thereof, wherein R³ represents alkyl.

5. The 2-dimethylamino-3-amido-6-amino-pyridine derivative according to claim 1, or a stereoisomer or a mixture of its stereoisomers, or a pharmaceutically-acceptable addition salt thereof, or an N-oxide thereof, wherein R³ represents phenyl, which phenyl is optionally substituted one or more times with substituents selected from alkyl, halo, alkoxy and trifluoromethyl.

6. The 2-dimethylamino-3-amido-6-amino-pyridine derivative according to claim 1, or a stereoisomer or a mixture of its stereoisomers, or a pharmaceutically-acceptable addition salt thereof, or an N-oxide thereof, wherein L represents a linker selected from —CR′R″—, —CR′R″—CH₂— and cycloalkyl, wherein R′ and R″, independently of each other, represent hydrogen, alkyl or halo.

7. The 2-dimethylamino-3-amido-6-amino-pyridine derivative according to claim 1, which is
N-[2-Dimethylamino-6-(4-fluoro-benzylamino)-pyridin-3-yl]-2-(3,4-difluoro-phenyl)-acetamide;
N-[2-Dimethylamino-6-(4-fluoro-benzylamino)-pyridin-3-yl]-2-(3,5-difluoro-phenyl)-acetamide;
N-[2-Dimethylamino-6-(4-fluoro-benzylamino)-pyridin-3-yl]-2-(3-fluoro-4-trifluoromethyl-phenyl)-acetamide;
(S)—N-[2-Dimethylamino-6-(4-fluoro-benzylamino)-pyridin-3-yl]-2-phenyl-propionamide;
N-[2-Dimethylamino-6-(4-fluoro-benzylamino)-pyridin-3-yl]-3-(3-fluoro-phenyl)-propionamide;
trans-2-Phenyl-cyclopropanecarboxylic acid[2-dimethylamino-6-(4-fluoro-benzylamino)-pyridin-3-yl]-amide;
N-[2-Dimethylamino-6-(4-fluoro-benzylamino)-pyridin-3-yl]-2-(3-fluoro-phenyl)-acetamide;
N-[2-Dimethylamino-6-(4-fluoro-benzylamino)-pyridin-3-yl]-3,3-dimethyl-butyramide;
or a stereoisomer or a mixture of its stereoisomers, or a pharmaceutically-acceptable addition salt thereof, or an N-oxide thereof.

8. The 2-dimethylamino-3-amido-6-amino-pyridine derivative according to claim 1, which is
2-(3,5-Difluoro-phenyl)-N-[2-dimethylamino-6-(2,4,6-trimethyl-benzylamino)-pyridin-3-yl]-acetamide;
2-(3,5-Difluoro-phenyl)-N-[2-dimethylamino-6-(2,6-dimethyl-benzylamino)-pyridin-3-yl]-acetamide;
N-[2-Dimethylamino-6-(5-fluoro-2-methyl-benzylamino)-pyridin-3-yl]-3,3-dimethyl-butyramide;
or a stereoisomer or a mixture of its stereoisomers, or a pharmaceutically-acceptable addition salt thereof, or an N-oxide thereof.

9. A pharmaceutical composition comprising a therapeutically effective amount of the 2-dimethylamino-3-amido-6-amino-pyridine derivative according to claim 1, or a stereoisomer or a mixture of its stereoisomers, or a pharmaceutically-acceptable addition salt thereof, or an N-oxide thereof.

10. (canceled)

11. (canceled)

12. (canceled)

13. (canceled)

14. (canceled)

15. (canceled)

16. A method of treatment, prevention or alleviation of a disease or a disorder or a condition of a living animal body, including a human, which disorder, disease or condition is responsive to activation of Kᵥ7 channels, which method comprises the step of administering to such a living animal body in need thereof, a therapeutically effective amount of the 2-dimethylamino-3-amido-6-amino-pyridine derivative according to claim 1, or a stereoisomer or a mixture of its stereoisomers, or a pharmaceutically-acceptable addition salt thereof, or an N-oxide thereof.

17. The use according to claim 11, wherein the disease, disorder or condition is pain, neurodegenerative disorders, migraine, bipolar disorders, mania, epilepsy, convulsions, seizures and seizure disorders, anxiety, depression, schizophrenia and urinary incontinence.

18. The use according to claim 11, wherein the disease, disorder or condition is pain, mild, moderate or severe pain, acute, chronic or recurrent pain, neuropathic pain, pain caused by migraine, postoperative pain, phantom limb pain, neuropathic pain, chronic headache, tension type headache, central pain, pain related to diabetic neuropathy, to post therapeutic neuralgia, or to peripheral nerve injury.
